(19) United States
(12) Patent Application Publication
(10) Pub. No.: US 2026/0272585 A1
(43) Pub. Date: Sep. 17, 2026

(54) SURGEON CONSOLE FOR A ROBOTIC SURGICAL SYSTEM

(71) Applicant: AURIS HEALTH, INC., Santa Clara, CA (US)

(72) Inventors: Neha Khemka, Lafayette, CA (US); Christian Ruiz, Liberty Hill, TX (US); Abhishek Sahasrabudhe, Santa Clara, CA (US); Jingyi Xu, Redwood City, CA (US); Marietta Johnson, Redwood City, CA (US)

(21) Appl. No.: 19/666,796

(22) Filed: May 4, 2026

Related U.S. Application Data
(63) Continuation of application No. PCT/IB2024/060981, filed on Nov. 6, 2024.
(60) Provisional application No. 63/596,563, filed on Nov. 6, 2023.

(51) Int. Cl. A61B 34/37; A61B 17/00; A61B 34/30; A61B 90/00

(52) U.S. Cl. CPC A61B 34/37; A61B 17/00; A61B 90/361; A61B 2017/00477; A61B 2034/301; A61B 2034/306

(57) ABSTRACT

Thermal cooling systems and methods for dissipating heat output by electronic components of a robotic surgical system. A system includes a thermal cooling system disposed within an interior space of an armrest console. The thermal cooling system includes a first haptic interface device component, a processing unit, a second haptic interface device component, a heat sink that receives heat generated by the processing unit during operation, and a plurality of fans. The plurality of fans are configured to pull air into the interior space, through the first haptic interface device component, through the heat sink, and through the second haptic interface device component.

### PRIORITY

This application is a continuation of International Application No. PCT/IB2024/060981, filed Nov. 6, 2024, entitled “SURGEON CONSOLE FOR A ROBOTIC SURGICAL SYSTEM,” which claims priority to U.S. Provisional Application No. 63/596,563, filed Nov. 6, 2023, entitled “SURGEON CONSOLE FOR A ROBOTIC SURGICAL SYSTEM,” the disclosures of which are incorporated by reference herein, in their entireties.

### TECHNICAL FIELD

This disclosure relates to systems and methods for robotic surgical systems, and more particularly to a surgeon console within a robotic surgical system.

### BACKGROUND

Robotic-assisted surgery is a rapidly advancing field that combines the precision of robotic machinery with the knowledge of skilled surgeons. A robotic-assisted surgical system typically includes a surgeon control console, one or more robotic arms, and a visualization system. The surgeon control console provides an interface for a surgeon to input commands that control the robotic arms and perform procedural tasks. The surgeon control console may be equipped with numerous electronic components which can create a relatively bulky housing. These bulky consoles can take up valuable space within an operating room and create a disconnected feel between the surgeon and the patient and operating room staff, making it difficult for a surgeon to communicate with staff or observe the procedure.

### BRIEF DESCRIPTIONS OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood with regard to the following description and accompanying drawings where:

FIG. 1 is a schematic block diagram illustrating some components of a robotic surgical system;

FIG. 2A is a schematic illustration of an aerial top-down view of a thermal cooling system disposed within an armrest console of a robotic surgical system;

FIG. 2B is a schematic illustration depicting forced airflow through a thermal cooling system disposed within an armrest console of a robotic surgical system;

FIG. 3A is a schematic illustration of an aerial top-down through-wall view of components of an assembly for processor cooling;

FIG. 3B is a schematic illustration of a cross-sectional view depicting the layering of components within an assembly for processor cooling;

FIG. 4A is a schematic illustration of an aerial top-down view of a heat sink assembly;

FIG. 4B is a perspective view of a heat sink assembly;

FIG. 5 is a perspective view of an armrest console of a robotic surgical system, wherein a portion of the housing of the armrest console is removed to reveal a thermal cooling system disposed therein;

FIG. 6 is a perspective view of an armrest console of a robotic surgical system, wherein two hook assemblies of the armrest console are pulled down into a partially open position;

FIG. 7 is a perspective view of internal components of a hook assembly for an armrest console of a robotic surgical system;

FIG. 8 is a perspective view of components of a hook assembly for an armrest console of a robotic surgical system;

FIGS. 9A-9C are schematic flow chart diagrams of a method for optimizing fan speeds within a thermal cooling system to maintain temperatures within a threshold temperature specification, and further to minimize noise output by cooling fans; and

FIG. 10 is a schematic block diagram illustrating components of an exemplary computing system.

### DETAILED DESCRIPTION

As discussed above, a surgeon control console may be equipped with numerous electronic components. It can be desirable to assemble these components within a relatively small housing, but these electronic components produce a significant amount of heat within the housing, and the heat should be dissipated to avoid damaging the electronic components. Thus, it is helpful to develop effective thermal management solutions to ensure the reliability and longevity of the surgeon control console.

Significant engineering challenges arise when seeking to dissipate heat generated by electronic components within a confined space. The housing of the surgeon control console may provide limited airflow, and this makes it difficult for air to passively cool the electronic components. Additionally, the surgeon control console typically includes multiple heat-generating components within a small space that can lead to localized hotspots that can damage delicate electronic and other components. Further, in many cases, a relatively small surgeon control console lacks the space for a traditional heat sink or other conventional cooling solution. Additionally, because the surgeon control console will be utilized during surgical procedures, it is desirable to ensure that conventional cooling methods, like fans, do not introduce excessive unwanted noise.

Disclosed herein are systems, methods, and devices for effective thermal management within a confined space. The systems, methods, and devices described herein may specifically be utilized within a surgeon control console of a robotic-assisted surgical system.

A robotic-assisted surgical system typically includes a robotic system comprising one or more robotic arms. A surgical instrument, such as an endoscope or surgical tool, may be affixed to each of the one or more robotic arms. The robotic-assisted surgical system additionally includes a visualization system. The visualization system includes image sensors, illuminations sources, and image signal processing components for outputting a real-time video stream of a surgical scene. The robotic-assisted surgical system additionally includes a surgeon control console that serves as the interface between a surgeon and the robotic-assisted surgical system. The surgeon control console enables the surgeon to control the robotic system and view the real-time video stream output by the visualization system.

The ergonomics of the surgeon control console are optimized to ensure the console is comfortable, easy-to-use, and does not introduce safety risks for the patient or healthcare personnel. Thus, to increase surgeon comfort and stability, the surgeon control console may be designed with an armrest portion. Control mechanisms for controlling the robotic-assisted surgical system may be disposed on, within, or around the armrest portion. Thus, the armrest portion is designed to ensure the surgeon may rest their arms and still easily access at least a portion of the control mechanisms. The control mechanisms may include, for example, one or more of a haptic interface device, haptic feedback device, controller, touchscreen interface, keyboard, mouse, voice recognition system, or any other component applicable for the purposes of this disclosure.

The control mechanisms for the robotic-assisted surgical system include numerous electronic components, including, for example, central processing units (CPUs), graphics processing units (GPUs), cables, microprocessors, sensors, and so forth. At least a portion of these electronic components may be disposed within an interior space defined by the armrest of the surgeon control console. Because the interior space of the armrest is space-constrained, and because the electronic components output significant heat during operation, it is necessary to incorporate efficient thermal cooling systems within the armrest of the surgeon control console.

Described herein are systems, methods, and devices for dissipating heat generated by electronic components within an armrest console of surgeon control console. At least a portion of the electronic components within the armrest console are in communication with a robotic surgical system, and data output by the electronic components is utilized to calculate one or more movements to be executed by the robotic surgical system. Specifically described herein is a thermal cooling system disposed within an interior space of the armrest console. The thermal cooling system is configured to dissipate heat generated by internal components of the armrest console, which includes a first haptic interface device component, a processing unit, and a second haptic interface device component. The thermal cooling system additionally includes a heat sink disposed adjacent to the processing unit, wherein the heat sink is configured to receive heat generated by the processing unit. The thermal cooling system additionally includes a plurality of fans configured to pull air into the interior space of the armrest console, and then pull air through each of the first haptic interface device, the heat sink, and the second haptic interface device. The plurality of fans are further configured to pull heated air out of the interior space of the armrest console.

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

Before the structure, systems, and methods are disclosed and described, it is to be understood that this disclosure is not limited to the particular structures, configurations, process steps, and materials disclosed herein as such structures, configurations, process steps, and materials may vary somewhat. It is also to be understood that the terminology employed herein is used for the purpose of describing particular embodiments only and is not intended to be limiting since the scope of the disclosure will be limited only by the appended claims and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element or step not specified in the claim.

As used herein, the phrase “consisting essentially of” and grammatical equivalents thereof limit the scope of a claim to the specified materials or steps and those that do not materially affect the basic and novel characteristic or characteristics of the claimed disclosure.

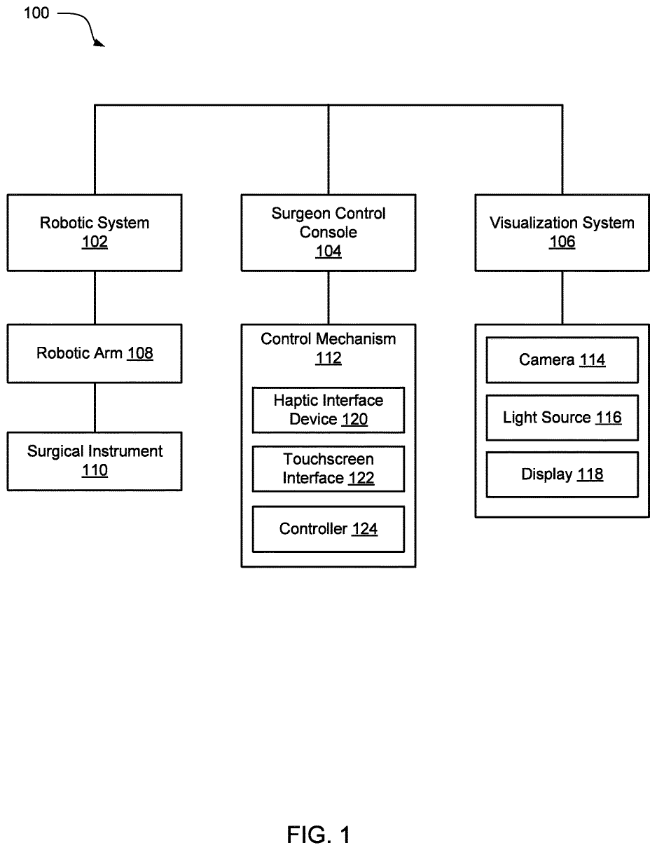

Referring now to the figures, FIG. 1 is a schematic block diagram illustrating some components of a system 100 for robotic surgery or robotic-assisted surgery. The system 100 includes robotic components and surgical instruments to assist surgeons in performing surgical procedures with enhanced precision and control. The system 100 may also be utilized to perform minimally invasive surgeries, although this is not required. In minimally invasive surgeries, the system 100 requires smaller incisions compared to traditional open surgery, which results in reduced trauma, less blood loss, smaller scars, and faster recovery times for patients. The system 100 includes capabilities for remote manipulation, such that a surgeon located remote from the system 100 can still instruct the system 100 on how to implement certain surgical procedures. The system 100 includes numerous interconnected components, including at least a robotic system 102, surgeon control console 104, and visualization system 106.

The robotic system 102 includes one or more robotic arms 108 each equipped with a surgical instrument 110. The robotic arms 108 are capable of providing increased precision and dexterity than human hands, which enables surgeons to perform intricate maneuvers with improved accuracy. The robotic system 102 includes one or more controllers configured to instruct the movements of the one or more of the robotic arms 108. Typically, each robotic arm includes a single tool receptacle and thus manages operations of a single surgical instrument 110.

In some embodiments, the robotic arms 108 are located at a surgical platform such as a table or bed and/or installed into a component of the system 100 itself. In some cases, the system 100 includes a column or other stable fixture configured to securely hold the robotic arms 108. In other configurations, the robotic arms 108 may be mounted on a cart, ceiling or sidewall, or other suitable support surface. The surgical instruments 110 may be installed and removed from the robotic arms 108 such that diverse types of surgical instruments 110 may be utilized by each of the robotic arms 108. A surgeon may select and request that certain surgical instruments 110 be installed in certain robotic arms 108 depending on the scope of the surgical procedure to be performed. The surgical instruments 110 may be removed and replaced during a surgical operation as needed. Additionally, the surgical instruments 110 may be removed for reprocessing and sterilization procedures. The surgical instruments 110 might include, for example, one or more of an endoscope comprising a camera, a retractor, a cauterizer, a stapler, a cutter, a scalpel, and so forth. The endoscope comprising the camera may have any suitable configuration depending on the surgical operation to be performed. For example, the endoscope may specifically be any of a laparoscope, laryngoscope, colonoscope, bronchoscope, sigmoidoscope, and so forth.

The surgeon control console 104 is in electronic communication with the robotic system 102 and the visualization system 106. The surgeon control console 104 includes one or more control mechanisms 112 that enable a user to provide instructions to be executed by the robotic system 102 and/or the visualization system 106. The surgeon control console 104 may be located remote from the robotic system 102 and the visualization system 106. In various implementations, the surgeon control console 104 could be located in the same operating room as the robotic system 102, located in an adjacent or nearby room, or teleoperated from a remote location in a different building, city, or country.

The control mechanism 112 may specifically include one or more of a haptic interface device (HID) 120, touchscreen interface 122, or controller 124. Additionally, the control mechanism 112 may include one or more of a foot pedal, voice recognition system, button, dial, keyboard, mouse, emergency stop, boundary control, and so forth. The control mechanisms 112 translate a surgeon's movements or voice commands into precise, scaled movements implemented by the robotic arms 108 and surgical instruments 110. The control mechanisms 112 may be constructed with built-in safety features to eliminate or reduce hand tremors and ensure the robotic system 102 executes the surgeon's controls with fluid motion. A user may utilize one or more of the available control mechanisms 112 based on preference and the scope of the procedure to be performed.

The surgeon control console 104 may specifically include an HID 120 that enables the surgeon to interact with a computer or other digital system through the sense of touch. The HID 120 provides haptic feedback configured to stimulate a user's sense of touch, texture, resistance, temperature, and/or vibration. The HID 120 thus enables a user to perceive physical sensations such as pressure, texture, vibration, or force while interacting with digital or remote objects. The HID 120 enables a surgeon to perform surgical movements with their arms and hands, and then have those surgical movements translated by the robotic system 102 into precise robotic movements. The HID 120 includes actuators and sensors. The actuators are the primary components that produce tactile sensations and may include, for example, vibration motors, piezoelectric actuators, and force feedback motors. The sensors detect the surgeon's interactions and movements, which are then translated into commands to be executed by the robotic system 102.

The one or more HIDs 120 utilized by the system 100 may include, for example, haptic gloves, haptic controllers, haptic vests or suits, haptic styluses or pens, or haptic displays. Haptic gloves include gloves equipped with sensors and actuators on the fingers and palm to provide users with a sense of touch and manipulation within a virtual environment. Haptic controllers may be implemented as a handheld device that incorporates haptic feedback mechanisms like rumble motors to simulate various sensations during use. Haptic vests or suits include wearable garments featuring embedded haptic actuators to cover the user's body and provide a comprehensive tactile experience. Haptic styluses or pens may be utilized in digital applications to provide feedback when drawing or writing on a digital surface. Haptic displays incorporate touch-sensitive surfaces that can simulate textures or provide feedback when interacting with on-screen elements.

The one or more HIDs 120 of the surgeon control console 104 may be utilized to receive input from a surgeon during a robotic-assisted surgery. For example, a surgeon may utilize haptic gloves to perform a virtual surgery, and then the surgeon's movements are translated to movements executed by the robotic system 102. Further for example, a surgeon may manipulate a haptic controller to indicate how certain robotic arms 108 or surgical instruments 110 should be moved, and then the surgeon's manipulations are translated to movements executed by the robotic system 102.

The surgeon control console 104 may further include the touchscreen interface 122. In some implementations, the touchscreen interface 122 is integrated into a housing of an armrest. The touchscreen interface 122 enables the surgeon to adjust various system settings applicable to any of the robotic system 102, the surgeon control console 104, or the visualization system 106. In some cases, the surgeon utilizes the touchscreen interface to request certain types of visualization data, such as color visualization, fluorescence visualization, multispectral visualization, and/or dimensional information.

The surgeon control console 104 may further include the controller 124, such as a joystick controller, button controller, or other controller. The controller 124 may include a handheld device that may be efficiently manipulated by a surgeon to provide precise movement instructions to the robotic system 102.

The surgeon control console 104 may specifically include one or more foot pedals (not shown in drawings). The foot pedals enable the surgeon to multitask without removing their hands from a primary control mechanisms 112 (such as, for example, the HID 120, touchscreen interface 122, controller 124, and so forth). The foot pedal may specifically be configured to control, for example, the visualization system 106, clutching of the surgical instruments 110, energy activation for cutting or coagulating tissue, switching between surgical instruments 110, and so forth.

The surgeon control console 104 may specifically include one or more safety controls, such as an emergency stop, a boundary control, or an override mechanism (not shown in drawings). The emergency stop is a control mechanism to immediately halt all robotic actions by the robotic system 102. The boundary control instructs the robotic system 102 to not move beyond pre-set limits, which may be selected to prevent unintended tissue damage. The override mechanism allows for immediate manual control or repositioning of the robotic arms 108 or surgical instruments 110.

The visualization system 106 enables the surgeon to view anatomical structures in detail and perform precise surgical interventions. The visualization system 106 includes one or more of a camera 114, light source 116, and display 118. The visualization system 106 may be executed by one or more processors that are dedicated to the visualization system 106 but communicate with either of the robotic system 102 and/or the surgeon control console 104. The visualization system 106 may be equipped with a plurality of light sources 116 that are independently actuatable and enable the camera 114 to capture color, fluorescence, or multispectral visualization data of a scene.

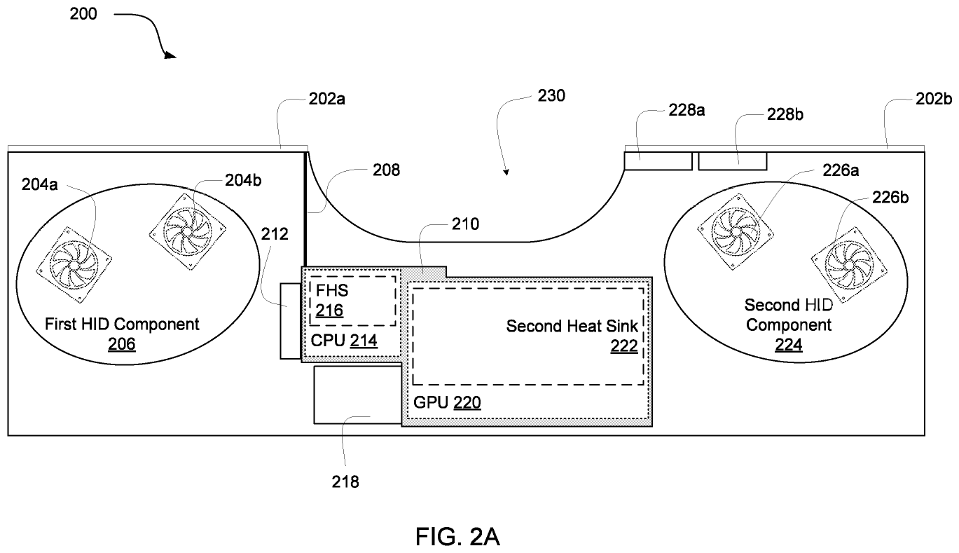

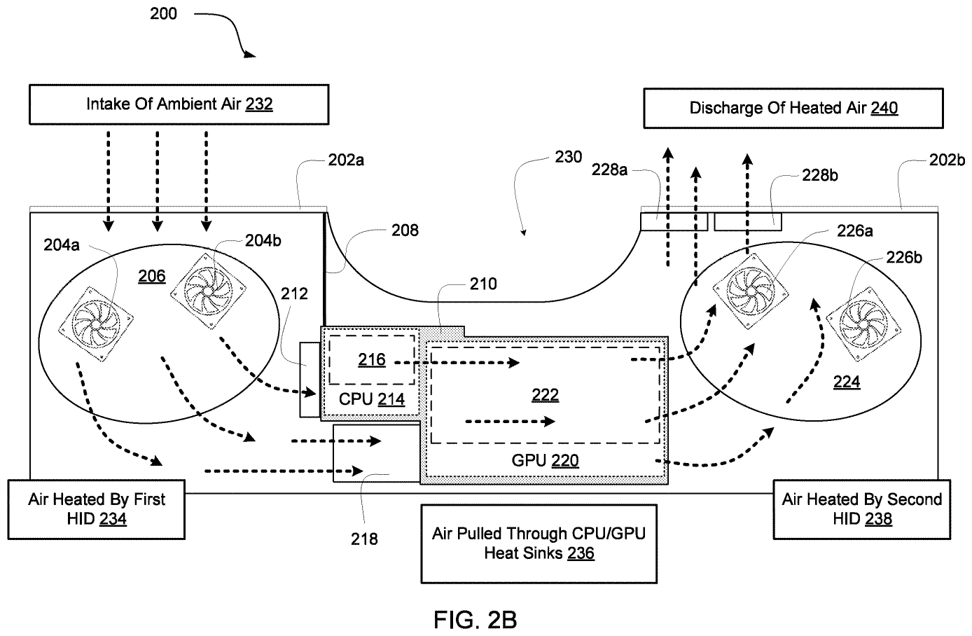

FIGS. 2A and 2B are schematic illustrations of an aerial top-down view of a thermal cooling system 200 that is configured to provide adequate cooling to a surgeon control console 104 while keeping noise levels low. FIGS. 2A and 2B each illustrate the components of the thermal cooling system, and FIG. 2B additionally illustrates airflow through the thermal cooling system 200.

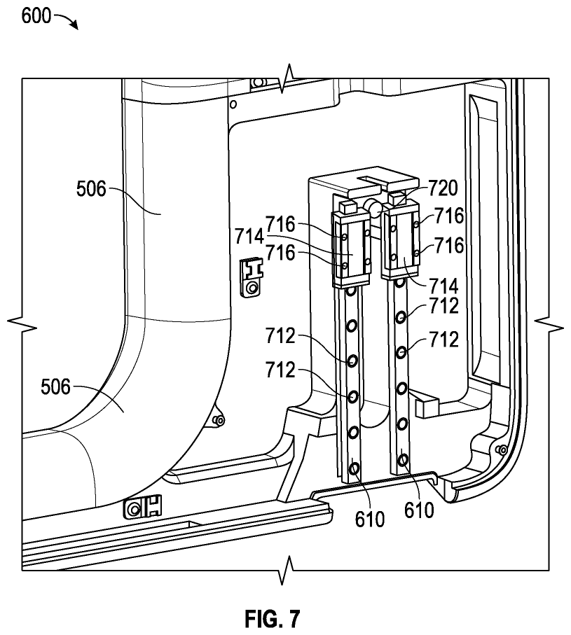

The thermal cooling system 200 is optimized to be disposed within an armrest console (see, for example, armrest console 500 first illustrated in FIG. 5) of the surgeon control console 104. The surgeon control console 104 includes a plurality of heat-generating components, including, for example, processors, displays, or components of the one or more control mechanisms 112. As shown in FIG. 2B, the thermal cooling system 200 is designed to efficiently capture and discharge heat generated by the electronic components of the surgeon control console 104. The thermal cooling system 200 is configured to maintain internal temperatures within safe and optimal operating temperature ranges to ensure the surgeon control console 104 can operate without damaging sensitive electronic components.

The thermal cooling system 200 is configured to capture, dissipate, and discharge heat generated by one or more of CPU(s), GPU(s), or components associated with the HIDs 120. In an embodiment, the thermal cooling system 200 is specifically configured to discharge heat generated by a first HID component 206, a second HID component 224, a CPU 214, and a GPU 220 as shown in FIGS. 2A-2B.

As shown in FIGS. 2A-2B, the thermal cooling system 200 may capture and dissipate heat generated by one or more HID components (see, for example, the first HID component 206 and the second HID component 224) that are disposed within an interior space defined by a housing of the surgeon control console 104. The HID components 206, 224 may include any component of the HID 120 and may specifically include an interface for establishing an electronic connection between the HID 120 and the processing unit of the surgeon control console 104. The HID components 206, 224 may include a structure or housing that is configured to encase electronic elements associated with the HID 120. These electronic elements may include, for example, interfaces for receiving a plug or cable providing direct electronic communication with an HID 120, processors for processing data received from the HID 120, memory devices, and so forth. The HID components 206, 224 may specifically include one or more processors configured to receive, translate, communicate, and/or assess sensor readings or instructions provided by the HID 120. The HID components 206, 224 disposed within the armrest console will generate significant amounts of heat, and this heat is discharged from the armrest console by the thermal cooling system 200 to maintain safe operating temperatures.

The thermal cooling system 200 may further capture and dissipate heat generated by one or more of a central processing unit (CPU) 214 or a graphics processing unit (GPU) 220. The CPU 214 and/or GPU 220 may be utilized to process inputs received from the one or more HIDs 120. Additionally, or alternatively, the CPU 214 and/or GPU 220 may be utilized to process operations for a computer that may include one or more control mechanisms 112 connected thereto.

The thermal cooling system 200 is disposed within an enclosed interior space. In an embodiment, the enclosed interior space is formed by a housing of the surgeon control console 104 and is specifically formed by the housing of an armrest console (see, for example, armrest console 500 first illustrated in FIG. 5). Because the housing of the armrest console defines a relatively small interior space, and because the combination of the HID component(s) 206, 224, the CPU(s) 214, and the GPU(s) 220 generate significant heat, it is necessary to implement an efficient thermal cooling system 200 to intake air and discharge heat outside the interior space of the surgeon console 104. It should be appreciated that the thermal cooling system 200 described herein fulfills stringent thermal and noise requirements for the surgeon control console 104.

The thermal cooling system 200 is enclosed within a housing that includes multiple vents 202a, 202b. The vents 202a, 202b may include holes or cutouts formed within a wall of the housing. The example illustrated in FIGS. 2A-2B specifically includes an intake vent 202a located on the left-hand side of the housing, and a discharge vent 202b located on the right-hand side of the housing. The airflow illustrated in FIG. 2B indicates that air is pulled into the housing through the intake vent 202a, and heated air is discharged from the housing through the discharge vent 202b. However, it should be appreciated that the direction of airflow may be reversed, and that air may pass directionally through all vents 202a, 202b.

The thermal cooling system 200 further includes a plurality of fans. In some implementations, the fans disposed within the thermal cooling system 200 are relatively small to ensure the cumulative noise output by the plurality of fans remains low. In the example illustrated in FIGS. 2A-2B, the thermal cooling system 200 includes a first HID fan 204a and a second HID fan 204b each associated with the first HID component 206. Additionally, the thermal cooling system 200 includes a first HID fan 226a and a second HID fan 226b associated with the second HID component 224. The HID fans 204a, 204b, 226a, 226b may be integrated within the housing formed for the HID components 206, 224.

The thermal cooling system 200 additionally includes fans that are not integrated within a housing of an HID component 206, 224 or otherwise associated with an HID component 206, 224. In the example illustrated in FIGS. 2A-2B, the thermal cooling system 200 includes a CPU fan 212 configured to pull air through the CPU 214 and/or a first heat sink 216 disposed above the CPU 214. In some embodiments, the CPU fan 212 includes a plurality of CPU fans 212 configured to pull air through the CPU 214 and/or the first heat sink 216. The thermal cooling system 200 includes a GPU fan 218 configured to pull air through the GPU 220 and/or a second heat sink 222 disposed above the GPU 220. In some embodiments, the GPU fan 218 includes a plurality of GPU fans 218 configured to pull air through the GPU 220 and/or the second heat sink 222. The thermal cooling system 200 further includes one or more discharge fans 228a, 228b configured to pull air out of the thermal cooling system 200 through the discharge vent 202b.

The thermal cooling system 200 includes a series of heat sinks that are disposed in between the first HID component 206 and the second HID component 224. Specifically, the thermal cooling system 200 includes a first heat sink 216 that is located above the CPU 214, and further includes a second heat sink 222 that is located above the GPU 220. A duct bracket 210 is layered over the first heat sink 216 and the second heat sink 222. The duct bracket 210 may include a singular piece of material that protects and encases each of the CPU 214, the first heat sink 216, the GPU 220, and the second heat sink 222. The duct bracket 210 ensures that heat generated by the CPU 214 and GPU 220 is efficiently pushed through the heat sinks 216, 222 and exits the housing through the discharge vent 202b. In the view illustrated in FIGS. 2A-2B, the CPU 214, the first heat sink 216, the GPU 220, and the second heat sink 222 are depicted with dotted lines to indicate they are not typically visible underneath the duct bracket 210. Notably, the relative positions and sizes of the CPU 214, the first heat sink 216, the GPU 220, and the second heat sink 222 are not drawn to scale in the schematic illustrations of FIGS. 2A-2B.

The thermal cooling system 200 additionally includes a baffle 208 that provides airflow path optimization to direct heated air through the heat sinks 216, 222. The baffle 208 forms a barrier between the first HID component 206 and the second HID component 226 so that air exiting the first HID component 206 is pushed through the heat sinks 216, 222 prior to exiting the housing through the discharge vent 202b.

In an embodiment, each of the fans, including the first and second HID fans 204a, 204b of the first HID component 206, the first and second HID fans 226a, 226b of the second HID component 224, the CPU fan 212, the GPU fan 218, and the discharge fans 228a, 228b is in a pull configuration. When all fans are in the pull configuration, airflow is optimized for efficiently cooling the electronic components and discharging heated air. This creates an airflow draft in the direction shown by the dotted arrowed lines in FIG. 2B. The fans 204a, 204b, 212, 218, 226a, 226b, 228a, 22b are speed controlled via firmware based on the temperatures of the electronics, including the first HID component 206, the second HID component 224, the CPU 214, and the GPU 220. The thermal cooling system 200 provides a margin high enough to run the fans 204a, 204b, 212, 218, 226a, 226b, 228a, 22b at a lower duty cycle and still maintain temperatures significantly below the slowdown temperature for the electronic components.

The placements of the various components within the thermal cooling system 200 are optimized to remove heat from the armrest console of the surgeon control console 104. The thermal cooling system 200 creates a ducted flow of air such that air is pulled into an interior space of the armrest console and is then pulled out of the interior space of the armrest console. This is shown in FIG. 2B, which illustrates an exemplary left-to-right airflow through an interior space defined by a housing of an armrest console.

In the example airflow illustrated in FIG. 2B, the thermal cooling process begins with an intake of air at 232. The air is pulled through the intake invent 202a by the first and second HID fans 204a, 204b of the first HID component 206. The air is then pulled through a housing of the first HID component 206 and is heated by the first HID component 206 at 234.

The air is then pulled through the CPU and GPU heat sinks at 236. Specifically, the CPU fan 212 pulls air into the first heat sink 216 associated with the CPU 214, and the GPU fan 218 pulls air into the second heat sink 222 associated with the GPU 220. The air is then heated by the second HID component 224 at 238. The air is pulled into the second HID component 224 by at least the first and second HID fans 226a, 226b of the second HID component 224. The heated air is then discharged from the interior space at 240. Specifically, one or more of the discharge fans 228a, 228b pulls the heated air out of the interior space by way of the discharge vent 202b. The fans on the right-hand side of FIG. 2B (see 226a, 226b, 228a, 228b) have the most significant impact on cooling electronic components within the armrest console because those fans are responsible for pulling air out of the armrest console.

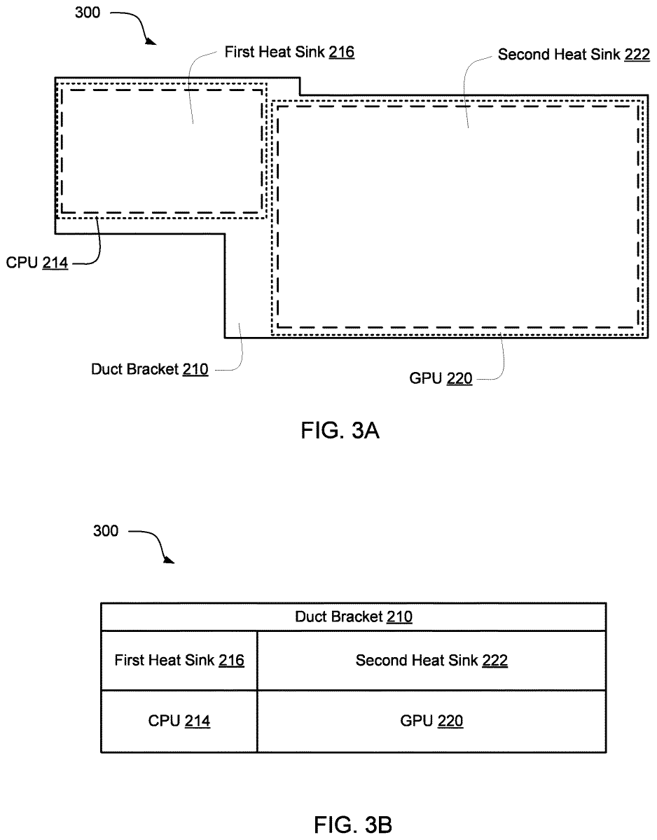

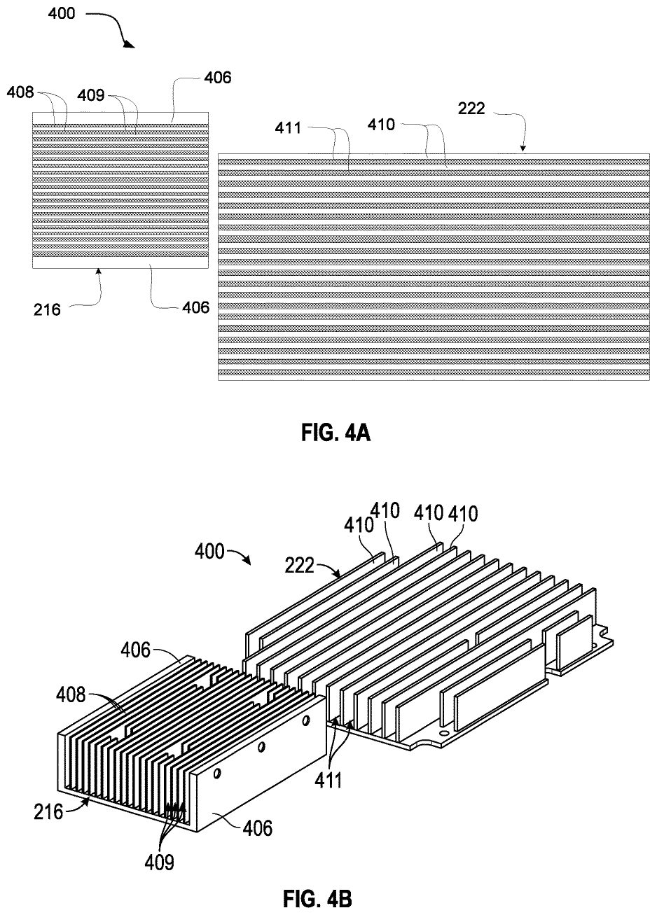

FIGS. 3A and 3B are schematic illustrations of an assembly 300 for processor cooling, and specifically for cooling the CPU 214 and GPU 220. FIG. 3A is an aerial top-down through-wall view of the assembly 300 that illustrates various components that are layered on top of each other. As shown in FIG. 3A, the duct bracket 210 (illustrated with a solid line) is the top-most component, and then the first and second heat sinks 216, 222 (illustrated with wide dotted lines) are located below the duct bracket 210, and then the CPU 214 and GPU 220 (illustrated with dotted lines) are located below the heat sinks 216, 222. FIG. 3B is a schematic cross-sectional view illustrating the layering of components within the assembly 300.

The heat sinks 216, 222 receive heat from the CPU 214 and the GPU 220 and then dissipate this heat into the surrounding air. The design of the heat sink fins, including the size, geometry, arrangement, and material, are optimized to maximize heat transfer density and reduce fluid flow resistance. This creates a path of less thermal resistance. As shown in FIGS. 3A-3B, the first heat sink 216 is primarily dedicated to receiving heat generated by the CPU 214, and the second heat sink 222 is primarily dedicated to receiving heat generated by the GPU 220.

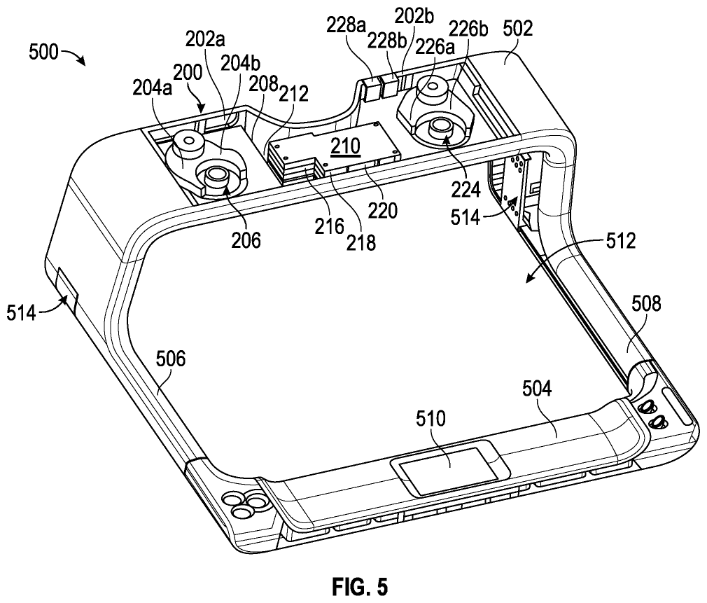

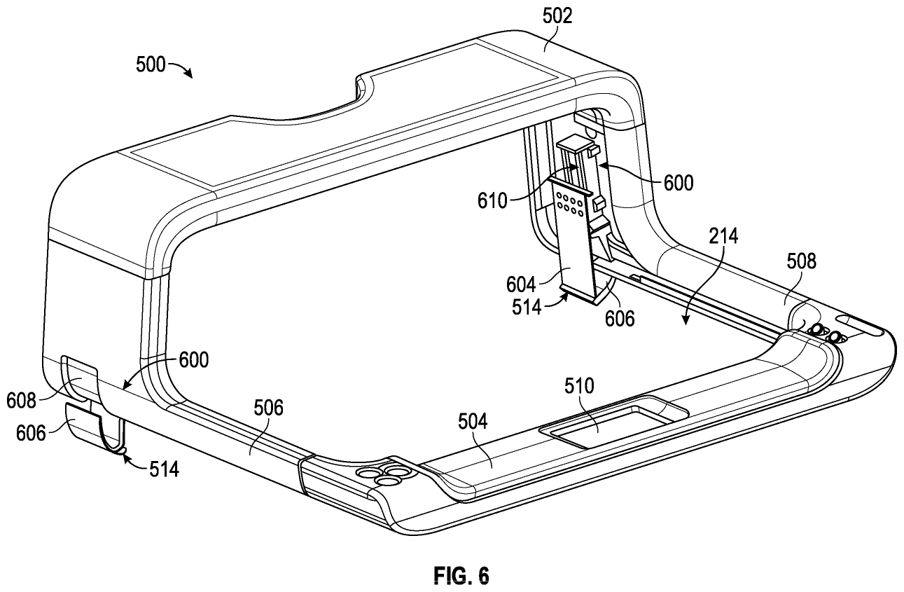

FIGS. 4A and 4B illustrate a heat sink assembly 400 including the first heat sink 216 and the second heat sink 222. FIG. 4A is an aerial top-down schematic illustration of the heat sink assembly 400. FIG. 4B is a perspective side view of the heat sink assembly 400.

The first heat sink 216 includes a plurality of first fins 408 and a plurality of first fin gaps 409 that are located in between the first fins 408. The second heat sink 222 includes a plurality of second fins 410 and a plurality of second fin gaps 411 that are located in between the second fins 410. In the schematic illustration of FIG. 4A, the solid components, including the first and second fins 408, 410 are illustrated in white, and the negative air space in between the solid components, including the first and second fin gaps 409, 411, are illustrated in grey.

In the example implementation illustrated in FIGS. 4A and 4B, the first heat sink 216 has a different configuration when compared with the second heat sink 222. Specifically, the first heat sink 216 includes a heat sink wall 406 disposed on a top side and a bottom side of the first heat sink 216. The heat sink wall 406 comprises a greater thickness than any of the first fins 408 of the first heat sink 216. The heat sink wall 406 may be machined with one or more holes disposed therethrough for mounting the first heat sink 216 within the assembly 400.

Additionally, the first heat sink 216 includes a different quantity of fins when compared with the second heat sink 222. In the example illustrated in FIGS. 4A-4B, the first heat sink 216 includes nineteen first fins 408 and two heat sink walls 406. By contrast, the second heat sink 222 includes sixteen second fins 410 and no heat sink walls. It should be appreciated that the quantity of fins within the first heat sink 216 or the heat sink 222 may be adjusted and optimized based on the implementation, without departing from the scope of the disclosure. In various implementations, the first heat sink 216 may include a different quantity of first fins 408 and may specifically include from about 16-22 fins. In various implementations, the second heat sink 222 may include a different quantity of second fins 410 and may specifically include from about 13-19 fins.

Additionally, the first heat sink 216 and the second heat sink 222 are configured with different fin widths and fin spacing. In the example illustrated in FIGS. 4A-4B, the first fins 408 of the first heat sink 216 have a narrower width than the second fins 410 of the second heat sink 222. Additionally, the first fin gaps 409 of the first heat sink 216 are narrower than the second fin gaps 411 of the second heat sink 222. It should be appreciated that the fin widths and fin spacing within the first heat sink 216 or the heat sink 222 may be adjusted and optimized based on the implementation, without departing from the scope of the disclosure.

In various implementations, the first fins 408 may be configured with different fin widths, and may specifically be configured with a fin width from about 0.4 mm to about 0.8 mm. Likewise, the first fin gaps 409 may vary depending on the implementation, and may specifically be from about 1.72 mm to about 2.72 mm. Further, the second fins 410 may be configured with different fin widths depending on the implementation, and may specifically be configured with a fin width from about 0.5 mm to about 1.5 mm. Likewise, the second fin gaps 411 may vary depending on the implementation, and may specifically be from about 4.2 mm to about 6.2 mm.

The variation in quantity of fins, fin width, and fin spacing between the first heat sink 216 and the second heat sink 222 aids in efficiently capturing and dissipating heat from the CPU 214 and the GPU 220. The heat sink assembly 400 provides an efficient thermal cooling system that enables the fans of the thermal cooling system 200 to effectively pull heated air out of an interior space within an armrest console. Additionally, due to the efficiency of the heat sink assembly 400, the fans of the thermal cooling system 200 are run at a relatively low speed to reduce noise levels. In some cases, the volume of the thermal cooling system 200 is maintained at 55 dBA or less even when the electronic components within the armrest console are operational.

The thermal cooling system 200 represents an improvement over conventional cooling systems. Other solutions may utilize a different quantity of fans and may cause some fans to operate in the push configuration while other fans operate in the pull configuration. This may introduce an inefficient airflow path that is not optimized for the interior space within an armrest console of a surgeon control console 104. Additionally, less efficient configurations with inefficient airflow pathways may necessitate that one or more of the fans be run at higher speeds. The higher fan speeds introduce excess and undesirable noise levels that may prevent a safety risk within a surgical environment.

The thermal cooling system 200 represents an improvement over alternative cooling solutions, including those utilizing liquid cooling systems or heat pipes. Liquid cooling systems circulate liquid through water blocks resting on top of processing chips. These systems require maintenance, can be very heavy, and are louder than the fan-based thermal cooling system 200 described herein. Heat pipe-based solutions utilize heat-transfer devices that combine the principles of thermal conductivity and phase transition to transfer heat between solid interfaces. However, heat pipe solutions are not ideal for space constrained spaces. Thus, these alternative cooling systems are not suitable for the armrest console of a robotic surgical system as discussed herein.

FIG. 5 is a perspective view of an armrest console 500 of a surgeon control console 104, and specifically illustrates wherein a portion of a housing of the armrest console 500 is removed to reveal the thermal cooling system 200 located within an interior space defined by the housing. As shown in FIG. 5, several components of the surgeon control console 104 are disposed within the interior space defined by the housing of the armrest console 500. Specifically, supporting electronic components for the one or more control mechanisms 112 are disposed within the interior space, including, for example, the HID components 206, 224, the CPU 214, and the GPU 220. Because the electronic components output significant heat, and because the interior space is relatively small, the armrest console 500 includes the thermal cooling system 200 to dissipate heat and prevent damage to the electronic components.

The armrest console 500 includes an upper platform 502 and an armrest 504. As shown in FIG. 5, the upper platform 502 (where the thermal cooling system 200 resides) is disposed opposite to the armrest 504 and is substantially parallel to the armrest 504. The armrest console 500 additionally includes a first sidearm 506 and a second sidearm 508 that are substantially parallel to one another. The armrest 504, upper platform 502, first sidearm 506, and second sidearm 508 collectively form a frame of the armrest console 500 and may collectively be referred to as the frame 502-508 herein.

The boundaries of the frame 502-508 define an interior area 512, and several components of the surgeon control console 104 may be disposed within the interior area 512. Typically, one or more control mechanisms 112 are disposed within the interior area. In a specific embodiment, a haptic interface device is disposed within the interior area 512, and a user may utilize the open interior area 512 to move their arms and hands while using the haptic interface device.

Each of the armrest 504, upper platform 502, first sidearm 506, and second sidearm 508 comprises a housing that defines an interior space, and numerous components may be disposed within the interior spaces defined by these components of the frame 502-508. Specifically, the armrest console 500 may include one or more of CPUs, GPUs, microcontrollers, field programmable gate arrays, cables, wires, interfaces, and other components disposed within the interior spaces of the frame 502-508.

The armrest console 500 includes a touchscreen interface 510 built into the armrest 504 housing. The touchscreen interface 510 enables a user to interact with the surgeon control console 104 directly without using a traditional input device like a mouse or keyboard. The touchscreen interface 510 may be configured with multi-touch capabilities to recognize multiple touch points simultaneously. In an example embodiment, the multi-touch capabilities enable a user to pinch or zoom to view different regions of an image rendered on a display. The touchscreen interface 510 may additionally be configured with gesture recognition to recognize patterns of touch, including swipes, taps, pinches, and other motions to execute certain commands. The touchscreen interface 510 may additionally be configured with haptic feed to provide tactile feedback, like vibrations, to acknowledge a touch or action.

The armrest console 500 additionally includes one or more stowable hooks 514 integrated into the housing of the armrest console 500. In FIG. 5, the hooks 514 are currently in the stowed or closed position, such that the hooks 514 are slid up into the first or second sidearm 506, 508 of the armrest console 500.

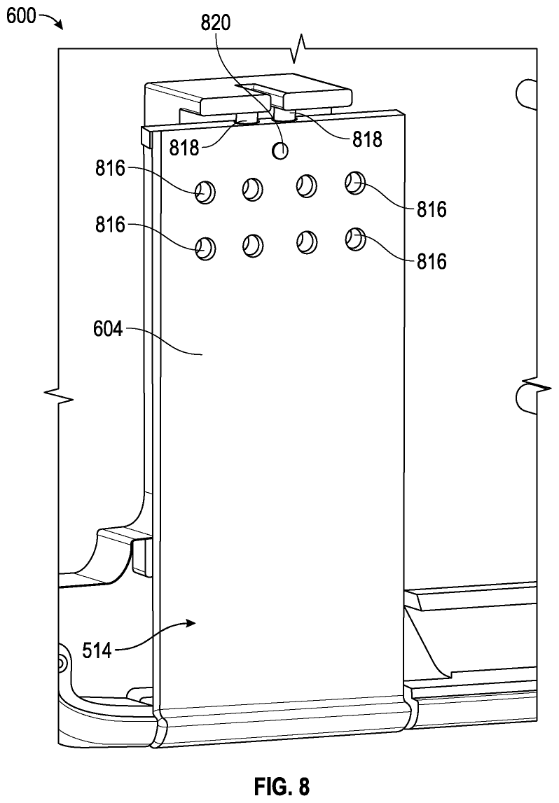

FIG. 6 is a perspective view of the armrest console 500. In contrast with the view illustrated in FIG. 5, the armrest console 500 illustrated in FIG. 6 includes a fully enclosed upper platform 502 such that the thermal cooling system 200 is not visible.

As shown in FIG. 6, the armrest console 500 may include one or more hook assemblies 600 and may specifically include a hook assembly 600 attached to the first sidearm 506, and another hook assembly 600 attached to the second sidearm 508. The hook assembly 600 provides for convenient storage of cables, including system connection cables and power connection cables associated with the system 100 for robotic surgery or robotic-assisted surgery. The hook assembly 600 prevents cables from getting in the way of surgical staff and allows for easier transport of the surgeon control console 104. Each of the two hook assemblies 600 integrated into the armrest console 500 is capable of holding at least 2.5 times the total mass of all cables intended to be used with the surgeon control console 104.

The hook assembly 600 includes the hook 514 that comprises a shank 604 and a bend 606. The length of the throat of the hook 514 (i.e., the length of the bend 606 from the bottom of the shank 604 to the top of the bend 606) is optimized for holding the cables intended to be used with the surgeon control console 104. Likewise, the length of the gape of the hook 514 (i.e., the distance between an inner wall of the bend 606 and an inner wall of the shank 604) is also optimized for holding the cables intended to be used with the surgeon control console 104.

Each hook assembly 600 additionally includes a hook cutout 608 cut into an exterior wall of the armrest console 500. The hook assembly 600 disposed on a first side of the armrest console 500 include a hook cutout 608 that is cut into an exterior wall of the first sidearm 506, as shown in FIG. 6. Likewise, the hook assembly 600 disposed on a second side of the armrest console 500 includes a hook cutout 608 that is cut into an exterior wall of the second sidearm 508 (not visible in FIG. 6). The geometry and depth of the hook cutout 608 corresponds with the geometry and thickness of the bend 606 of the hook 514. This ensures that the hook 514 may be stowed away to form a smooth integration with the housing of the armrest console 500, as shown at least in FIG. 5.

Each hook assembly 600 additionally includes a linear rail 610. The linear rail 610 is integrated into the first sidearm 506 and the second sidearm 508 of the armrest console 500. The linear rail 610 comprises one or more rigid rods for holding the shank 604 of the hook 514 and enabling the hook 514 to slide up and down. When the hook 514 is fully slid up the linear rail 610, the hook 514 is disposed within a closed or stowed position. When the hook 514 is fully slid down the linear rail 610, the hook 514 is disposed within an open position and is ready to carry cables for the surgeon control console 104.

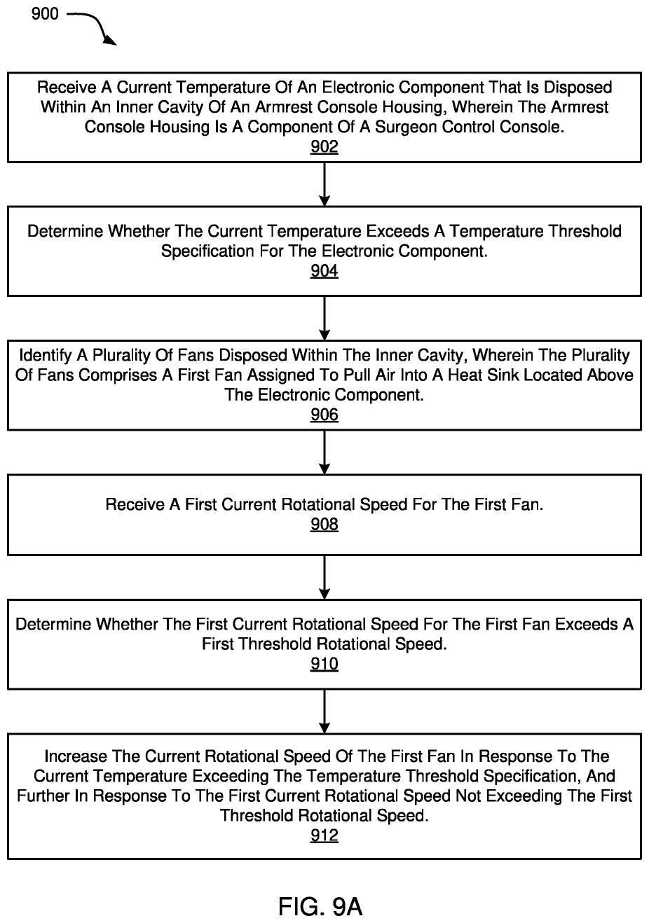

FIG. 7 is a perspective view of components of the hook assembly 600, and specifically illustrates components disposed within an interior space defined by the housing of the armrest console 500. As shown in FIG. 7, the linear rails 610 are secured to the housing of the armrest console 500, and thus enable the hook 514 to slide up and down relative to the housing of the armrest console 500.

The linear rails 610 are attached to the first sidearm 506 or the second sidearm 508 by way of a plurality of fasteners 712, which may specifically include machined screws or another fastening mechanism. The hook assembly 600 additionally includes one or more sliding blocks 714. Each of the one or more linear rails 610 includes a sliding block 714 capable of sliding up and down the linear rail 610. The sliding block 714 may include ball bearings to ensure smooth movement up and down the linear rail 610. Additionally, the sliding block 714 may include guardrails to stabilize movement and prevent twisting when sliding up and down the linear rail 610. The sliding block 714 includes a plurality of fastening holes 716 disposed therethrough. In some cases, the fastening holes 716 are threaded and configured to securely and releasably hold a machined screw. In the example illustrated in FIG. 7, each of the two sliding blocks 714 includes four fastening holes 716.

The hook assembly 600 additionally includes a magnet 720 disposed at the proximal end of the hook assembly 600. The magnet 720 interfaces with a corresponding magnet or magnetic material on the hook 514. The magnet 720 thereby maintains the hook 514 in the stowed or closed position when the hook 514 is stowed away and not being utilized to carry cables for the surgeon control console 104.

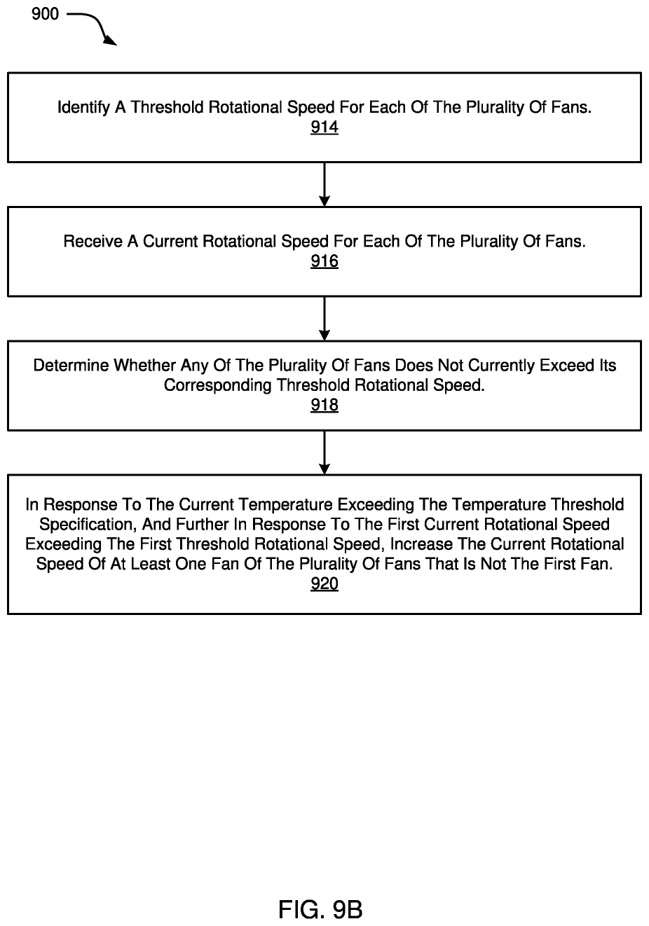

FIG. 8 is a perspective view of components of the hook assembly 600, and specifically illustrates wherein the shank 604 of the hook 514 is fully slid up the linear rails 610 (not visible in FIG. 8) and is disposed in a stowed or closed position.

The shank 604 includes a plurality of fastening holes 816 disposed therethrough. The plurality of fastening holes 816 correspond with the fastening holes 716 of the sliding blocks 714. The corresponding fastening holes 716, 816 may be threaded such that a machined screw may be screwed through the corresponding fastening holes 716, 816 to secure the shank 604 to the sliding blocks 714. In the example illustrated in FIG. 8, the shank 604 includes eight fastening holes 816 corresponding with the two pairs of four fastening holes 716 associated with the two sliding blocks 714 illustrated in FIG. 7.

The shank 604 includes a magnet 820 and/or magnetic material. The magnet 820 and/or magnetic material is positioned to interact with the magnet 720 when the hook 514 is slip up the linear rails 610 into the stowed position.

The hook assembly 600 additionally includes a bumper 818. The bumper 818 is located at a proximal end of the hook assembly 600 and is configured to dampen the sound of the sliding block 714 reaching the proximal end of the hook assembly 600 when the hook 514 is moved into the stowed or closed position. In an embodiment, the hook assembly 600 includes four or more bumpers 818 each configured to dampen the sound of sliding the shank 604 of the hook assembly 600 into the stowed position.

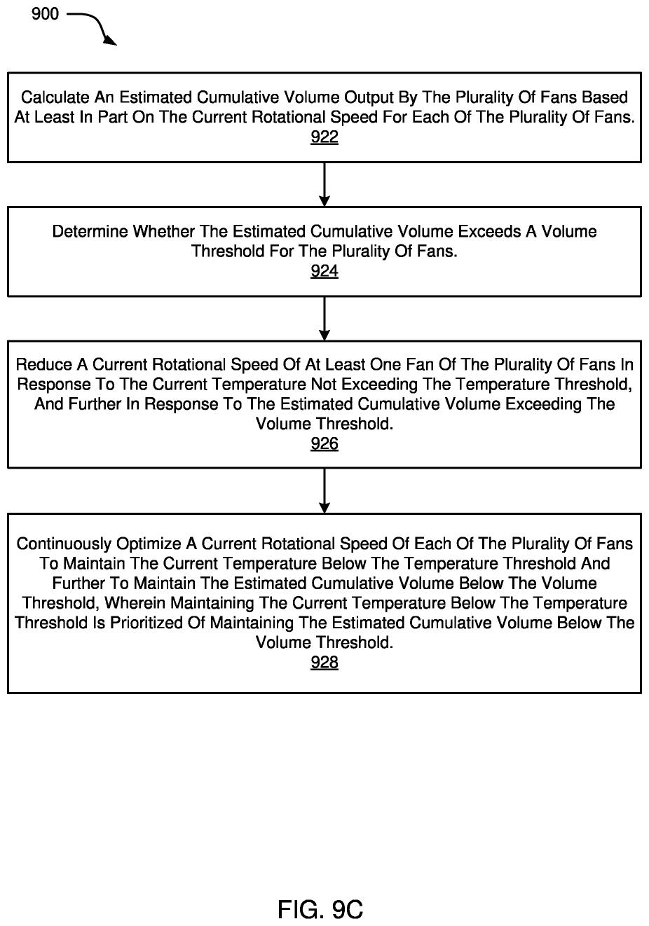

FIGS. 9A-9C are schematic flow chart diagrams of a method 900 for controlling heat within an armrest console 500 of a surgeon control console 104. The method 900 may be executed by a processor disposed within the surgeon control console 104.

The method 900 includes receiving at 902 a current temperature of an electronic component that is disposed within an inner cavity of an armrest console 500 housing. The armrest console 500 may specifically be a component of a surgeon control console 104 within a system 100 for robotic surgery or robotic-assisted surgery. The temperature may be output by a temperature sensor associated with an electronic component, such as the first or second HID component 206, 224, the CPU 214, or the GPU 220. The method 900 includes determining at 904 whether the current temperature exceeds a temperature threshold specification for the electronic component. The temperature threshold specification for the electronic component may be provided by a manufacturer of the electronic component to indicate when the electronic component will slow down, become less efficient, or begin to degrade due to high temperatures.

The method 900 includes identifying at 906 a plurality of fans disposed within the inner cavity, wherein the plurality of fans comprises a first fan assigned to pull air into a heat sink located above the electronic component. The first fan may specifically include the CPU fan 212 or the GPU fan 218 first described in connection with FIGS. 2A-2B. The method 900 includes receiving at 908 a first current rotational speed for the first fan.

The method 900 includes determining at 910 whether the first current rotational speed for the first fan exceeds a first threshold rotational speed. The first threshold rotational speed may be predetermined based on the noise output of the first fan at various rotational speeds. For example, a user may determine that the first fan is too loud at certain speeds, and then set the first threshold rotational speed based on the first fan's noise output.

The method 900 includes increasing at 912 the current rotational speed of the first fan in response to the current temperature exceeding the temperature threshold specification, and further in response to the first current rotational speed not exceeding the first threshold rotational speed. Thus, the rotational speed of the first fan may be continuously increased up to the first threshold rotational speed. The rotational speed of the first fan may be optimized and increased based on the current temperature of the electronic component.

The method 900 includes identifying at 914 a threshold rotational speed for each of the plurality of fans. In an embodiment, each fan of the thermal cooling system 200 may have a unique threshold rotational speed. The method 900 includes receiving at 916 a current rotational speed for each of the plurality of fans. Each fan of the thermal cooling system 200 could have a different current rotational speed at a given time. The method 900 includes determining at 918 whether any of the plurality of fans does not currently exceed its corresponding threshold rotational speed. The determining at 918 thus includes determining the speed of any of the plurality of fans could be increased without exceeding the corresponding threshold rotational speeds.

The method 900 includes increasing at 920 the current rotational speed of at least one fan of the plurality of fans in response to the current temperature of the electronic component exceeding the temperature threshold specification, and further in response to the first current rotational speed exceeding the first threshold rotational speed.

The method 900 includes calculating at 922 an estimated cumulative volume output by the plurality of fans based at least in part on the current rotational speed for each of the plurality of fans. The calculating at 922 may be performed based on data stored in memory that indicates the estimated volume of each fan at a variety of different rotational speeds. The calculating at 922 may alternatively be performed based on data output by a microphone that continuously monitors the noise output of the fans.

The method 900 includes determining at 924 whether the estimated cumulative volume exceeds a volume threshold for the plurality of fans. The method 900 includes reducing at 926 a current rotational speed of at least one fan of the plurality of fans in response to the current temperature not exceeding the temperature threshold specification, and further in response to the estimated cumulative volume exceeding the volume threshold. The method 900 includes continuously optimizing at 928 a current rotational speed of each of the plurality of fans to maintain the current temperature below the temperature threshold and further to maintain the estimated cumulative volume below the volume threshold, wherein maintaining the current temperature below the temperature threshold specification is prioritized over maintaining the estimated cumulative volume below the volume threshold.

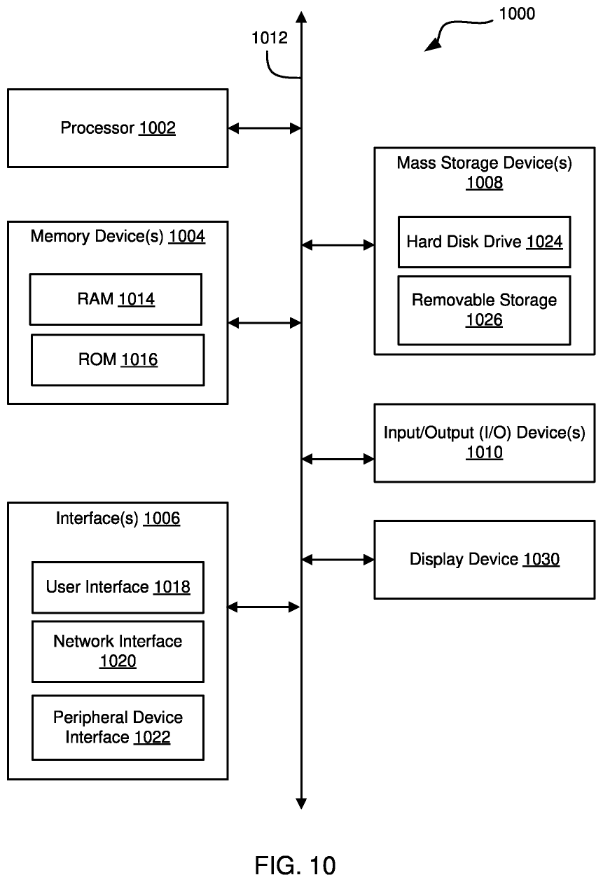

FIG. 10 illustrates a schematic block diagram of an example computing device 1000. The computing device 1000 may be used to perform various procedures, such as those discussed herein. The computing device 1000 can perform various monitoring functions as discussed herein, and can execute one or more application programs, such as the application programs or functionality described herein. The computing device 1000 can be any of a wide variety of computing devices, such as a desktop computer, in-dash computer, vehicle control system, a notebook computer, a server computer, a handheld computer, tablet computer and the like.

The computing device 1000 includes one or more processor(s) 1004, one or more memory device(s) 1004, one or more interface(s) 1006, one or more mass storage device(s) 1008, one or more Input/output (I/O) device(s) 1010, and a display device 1030 all of which are coupled to a bus 1012. Processor(s) 1004 include one or more processors or controllers that execute instructions stored in memory device(s) 1004 and/or mass storage device(s) 1008. Processor(s) 1004 may also include several types of computer-readable media, such as cache memory.

Memory device(s) 1004 include various computer-readable media, such as volatile memory (e.g., random access memory (RAM) 1014) and/or nonvolatile memory (e.g., read-only memory (ROM) 1016). Memory device(s) 1004 may also include rewritable ROM, such as Flash memory.

Mass storage device(s) 1008 include various computer readable media, such as magnetic tapes, magnetic disks, optical disks, solid-state memory (e.g., Flash memory), and so forth. As shown in FIG. 10, a particular mass storage device 1008 is a hard disk drive 1024. Various drives may also be included in mass storage device(s) 1008 to enable reading from and/or writing to the various computer readable media. Mass storage device(s) 1008 include removable media 1026 and/or non-removable media.

I/O device(s) 1010 include various devices that allow data and/or other information to be input to or retrieved from computing device 1000. Example I/O device(s) 1010 include cursor control devices, keyboards, keypads, microphones, monitors or other display devices, speakers, printers, network interface cards, modems, and the like.

Display device 1030 includes any type of device capable of displaying information to one or more users of computing device 1000. Examples of display device 1030 include a monitor, display terminal, video projection device, and the like.

Interface(s) 1006 include various interfaces that allow computing device 1000 to interact with other systems, devices, or computing environments. Example interface(s) 1006 may include any number of different network interfaces 1020, such as interfaces to local area networks (LANs), wide area networks (WANs), wireless networks, and the Internet. Other interface(s) include user interface 1018 and peripheral device interface 1022. The interface(s) 1006 may also include one or more user interface elements 1018. The interface(s) 1006 may also include one or more peripheral interfaces such as interfaces for printers, pointing devices (mice, track pad, or any suitable user interface now known to those of ordinary skill in the field, or later discovered), keyboards, and the like.

Bus 1012 allows processor(s) 1004, memory device(s) 1004, interface(s) 1006, mass storage device(s) 1008, and I/O device(s) 1010 to communicate with one another, as well as other devices or components coupled to bus 1012. Bus 1012 represents one or more of several types of bus structures, such as a system bus, PCI bus, IEEE bus, USB bus, and so forth.

For purposes of illustration, programs and other executable program components are shown herein as discrete blocks, although it is understood that such programs and components may reside at various times in different storage components of computing device 1000 and are executed by processor(s) 1002. Alternatively, the systems and procedures described herein, including programs or other executable program components, can be implemented in hardware, or a combination of hardware, software, and/or firmware. For example, one or more application specific integrated circuits (ASICs) can be programmed to conduct one or more of the systems and procedures described herein.

### EXAMPLES

The following examples pertain to preferred features of further embodiments:
Example 1 is a system. The system includes a housing that defines an interior space. The system includes a thermal cooling system disposed within the interior space defined by the housing. The thermal cooling system includes a processing unit that generates heat during operation and a haptic interface device component that generates heat during operation. The thermal cooling system further includes a first fan configured to pull ambient air into the interior space, a heat sink that receives the heat generated by the processing unit during operation, and a second fan configured to pull heated air out of the interior space.
Example 2 is a system as in Example 1, wherein the housing comprises a portion of an armrest console, and wherein the armrest console is a component of a surgeon control console for providing instructions to a robotic surgical system.
Example 3 is a system as in any of Examples 1-2, wherein the processing unit comprises a central processing unit (CPU) and a graphics processing unit (GPU), and wherein the heat sink receives the heat generated by one or more of the CPU or the GPU.
Example 4 is a system as in any of Examples 1-3, wherein the heat sink comprises: a first heat sink disposed adjacent to the CPU, wherein the first heat sink primarily receives the heat generated by the CPU during operation; and a second heat sink disposed adjacent to the GPU, wherein the second heat sink primarily receives the heat generated by the GPU during operation.
Example 5 is a system as in any of Examples 1-4, wherein the first heat sink comprises a first plurality of heat sink specifications comprising: a first plurality of fins, wherein the first heat sink comprises a first quantity of the first plurality of fins; a first fin width, wherein at least a portion of the first plurality of fins has the first fin width; and a first fin spacing, wherein two or more of the first plurality of fins is spaced apart according to the first fin spacing; and wherein the second heat sink comprises a second plurality of heat sink specifications comprising: a second plurality of fins, wherein the second heat sink comprises a second quantity of the second plurality of fins; a second fin width, wherein at least a portion of the second plurality of fins has the second fin width; and a second fin spacing, wherein two or more of the second plurality of fins is spaced apart according to the second fin spacing; and wherein at least one of the first plurality of heat sink specifications is different from a corresponding specification of the second plurality of heat sink specifications.
Example 6 is a system as in any of Examples 1-5, wherein the first quantity of the first plurality of fins is different than the second quantity of the second plurality of fins.
Example 7 is a system as in any of Examples 1-6, wherein the first fin width is different than the second fin width.
Example 8 is a system as in any of Examples 1-7, wherein the first fin spacing is different than the second fin spacing.
Example 9 is a system as in any of Examples 1-8, wherein the haptic interface device component comprises two or more haptic interface device components, and wherein the interior space defined by the housing comprises: a first side and a second side, wherein the second side is located opposite to the first side; a first vent disposed at the first side, wherein the first fan is configured to pull the ambient air into the interior space by way of the first vent; a first haptic interface device component disposed at the first side; a second haptic interface device component disposed at the second side; and a second vent disposed at the second side, wherein the second fan is configured to pull the heated air out of the interior space by way of the second vent.
Example 10 is a system as in any of Examples 1-9, wherein the heat sink is disposed in between the first haptic interface device component and the second haptic interface component such that the ambient air pulled into the interior space passes through the heat sink.
Example 11 is a system as in any of Examples 1-10, wherein the heat sink is disposed above the processing unit such that the processing unit is disposed in between the first haptic interface device component and the second haptic interface device component, and wherein the heated air is heated at least in part by the heat generated by the processing unit.
Example 12 is a system as in any of Examples 1-11, further comprising a baffle disposed within the interior space defined by the housing, wherein the baffle prevents the ambient air pulled into the interior space from moving to the second side prior to passing through the heat sink.
Example 13 is a system as in any of Examples 1-12, wherein the first fan is a first haptic interface device fan that is disposed within the first haptic interface device component; and wherein the second fan is a second haptic interface device fan that is disposed within the second haptic interface device component.
Example 14 is a system as in any of Examples 1-13, wherein the thermal cooling system further comprises: a central processing unit (CPU) fan configured to pull air into a first heat sink disposed above a CPU; a graphics processing unit (GPU) fan configured to pull air into a second heat sink disposed above a GPU; and an exit vent fan disposed adjacent to the second vent, wherein the exit vent fan is also configured to pull the heated air out of the interior space by way of the second vent.
Example 15 is a system as in any of Examples 1-14, wherein the haptic interface device component comprises a processor in communication with a surgical haptic interface device; wherein the surgical haptic interface device is disposed outside the interior space defined by the housing; and wherein the processor of the haptic interface device component is configured to translate movements of the surgical haptic interface device into robotic movements executed by a robotic surgical system.
Example 16 is a system as in any of Examples 1-15, wherein the housing comprises a portion of an armrest console; wherein the armrest console is a component of a surgeon control console for providing instructions to a robotic surgical system; and wherein the robotic surgical system further comprises: a robotic system comprising a robotic arm and a surgical instrument attached to the robotic arm; and a visualization system comprising a camera and a light source.
Example 17 is a system as in any of Examples 1-16, wherein the housing is a component of an upper platform of an armrest console, and wherein the armrest console further comprises: an armrest disposed substantially parallel to the upper platform; a first sidearm attached to the upper platform and the armrest, wherein the first sidearm is substantially perpendicular to the upper platform and the armrest; and a second sidearm attached to the upper platform and the armrest, wherein the second sidearm is disposed substantially parallel to the first sidearm.
Example 18 is a system as in any of Examples 1-17, further comprising one or more hook assemblies, wherein each of the one or more hook assemblies is attached to the first sidearm or the second sidearm, and wherein each of the one or more hook assemblies comprises: a hook comprising a hook shank and a hook bend; a linear rail; and a sliding block configured to slide along the linear rail; wherein the hook shank is attached to the sliding block.
Example 19 is a system as in any of Examples 1-18, wherein each of the one or more hook assemblies further comprises a hook cutout configured to receive the hook shank, wherein the hook cutout is cut into a sidewall of one or more of the first sidearm or the second sidearm.
Example 20 is a system as in any of Examples 1-19, wherein each of the one or more hook assemblies further comprises: a first magnet attached to the linear rail at a proximal end of the linear rail, wherein the proximal end of the linear rail is disposed nearer to the thermal cooling system; and one or more of a second magnet or a magnetic material attached to the hook shank at a proximal end of the hook shank, wherein the proximal end of the hook shank is disposed nearer to the thermal cooling system; wherein the first magnet does not engage with the one or more of the second magnet or the magnetic material when the hook shank is dropped into an open position; and wherein the first magnet engages with the one or more of the second magnet or the magnetic material when the hook shank is disposed within the hook cutout in a closed position.
Example 21 is a system for thermal cooling within a surgeon control console of a robotic surgical system. The system includes a housing of the surgeon control console, wherein the housing defines an interior space. The system includes a thermal cooling system disposed within the interior space defined by the housing. The thermal cooling system includes a first haptic interface device component, a processing unit, a second haptic interface device component, a heat sink that receives heat generated by the processing unit during operation, and a plurality of fans. The plurality of fans is configured to pull ambient air into the interior space, through the first haptic interface device component, through the heat sink, and through the second haptic interface device component.
Example 22 is a system as in Example 21, wherein the interior space defined by the housing is disposed within an armrest console of the surgeon control console, and wherein the surgeon control console provides data to the robotic surgical system that results in the robotic surgical system moving one or more robotic arms or surgical instruments.
Example 23 is a system as in any of Examples 21-22, wherein the processing unit comprises a central processing unit (CPU) and a graphics processing unit (GPU).
Example 24 is a system as in any of Examples 21-23, wherein the heat sink comprises: a first heat sink located adjacent to the CPU, wherein the first heat sink primarily receives heat generated by the CPU during operation; and a second heat sink located adjacent to the GPU, wherein the second heat sink primarily receives heat generated by the GPU during operation.
Example 25 is a system as in any of Examples 21-24, wherein the first heat sink comprises a first plurality of heat sink specifications comprising: a first plurality of fins, wherein the first heat sink comprises a first quantity of the first plurality of fins; a first fin width, wherein at least a portion of the first plurality of fins has the first fin width; and a first fin spacing, wherein two or more of the first plurality of fins is spaced apart according to the first fin spacing; and wherein the second heat sink comprises a second plurality of heat sink specifications comprising: a second plurality of fins, wherein the second heat sink comprises a second quantity of the second plurality of fins; a second fin width, wherein at least a portion of the second plurality of fins has the second fin width; and a second fin spacing, wherein two or more of the second plurality of fins is spaced apart according to the second fin spacing; and wherein at least one of the first plurality of heat sink specifications is different from a corresponding specification of the second plurality of heat sink specifications.
Example 26 is a system as in any of Examples 21-25, wherein the first quantity of the first plurality of fins is different than the second quantity of the second plurality of fins.
Example 27 is a system as in any of Examples 21-26, wherein the first fin width is different than the second fin width.
Example 28 is a system as in any of Examples 21-27, wherein the first fin spacing is different than the second fin spacing.
Example 29 is a system as in any of Examples 21-28, and wherein the interior space defined by the housing comprises: a first side and a second side, wherein the second side is located opposite to the first side; a first vent disposed at the first side, wherein a first fan of the plurality of fans is configured to pull the ambient air into the interior space by way of the first vent; the first haptic interface device component disposed at the first side; the second haptic interface device component disposed at the second side; and a second vent disposed at the second side, wherein a discharge fan of the plurality of fans is configured to pull heated air out of the interior space by way of the second vent.
Example 30 is a system as in any of Examples 21-29, wherein the processing unit and the heat sink are disposed in between the first haptic interface device component and the second haptic interface component such that an airflow pathway for the thermal cooling system comprises: the ambient air entering the interior space through the first vent; the ambient air passing through the first haptic interface device component to generate heated air; the heated air passing through the heat sink after passing through the first haptic interface device component; the heated air passing through the second haptic interface device component after passing through the heat sink; and the heated air exiting the interior space through the second vent after passing through the second haptic interface device component.
Example 31 is a system as in any of Examples 21-30, wherein the processing unit and the heat sink comprises: a central processing unit (CPU) and a first heat sink disposed above the CPU; and a graphics processing unit (GPU) and a second heat sink disposed above the GPU; wherein the heated air passing through the heat sink comprises the heated air passing through the first heat sink disposed above the CPU or the second heat sink disposed above the GPU.
Example 32 is a system as in any of Examples 21-31, further comprising a baffle disposed within the interior space defined by the housing, wherein the baffle prevents the ambient air pulled into the interior space from moving to the second side prior to passing through the heat sink.
Example 33 is a system as in any of Examples 21-32, wherein the plurality of fans comprises: a first plurality of haptic interface device component fans associated with the first haptic interface device component; and a second plurality of haptic interface device component fans associated with the second haptic interface device component.
Example 34 is a system as in any of Examples 21-33, wherein the plurality of fans further comprises: a central processing unit (CPU) fan configured to pull air into a first heat sink disposed above a CPU; a graphics processing unit (GPU) fan configured to pull air into a second heat sink disposed above a GPU; and a discharge fan configured to pull heated air out of the interior space by way of a vent cut into a wall of the housing.
Example 35 is a system as in any of Examples 21-34, wherein one of more of the first haptic interface device component or the second haptic interface device component comprises a processor in communication with a surgical haptic interface device; wherein the surgical haptic interface device is disposed outside the interior space defined by the housing; and wherein the processor in communication with the surgical haptic interface device is configured to translate movements of the surgical haptic interface device into robotic movements executed by the robotic surgical system.
Example 36 is a system as in any of Examples 21-35, wherein the robotic surgical system comprises: a robotic system comprising a robotic arm and a surgical instrument attached to the robotic arm; the surgeon control console in communication with the robotic system, wherein the surgeon control console provides data to the robotic system, and wherein the robotic system utilizes the data to calculate one or more movements to be executed by the robotic arm or the surgical instrument; and a visualization system comprising a camera and a light source.
Example 37 is a system as in any of Examples 21-36, wherein the housing is a component of an upper platform of an armrest console, and wherein the armrest console further comprises: an armrest disposed substantially parallel to the upper platform; a first sidearm attached to the upper platform and the armrest, wherein the first sidearm is substantially perpendicular to the upper platform and the armrest; and a second sidearm attached to the upper platform and the armrest, wherein the second sidearm is disposed substantially parallel to the first sidearm.
Example 38 is a system as in any of Examples 21-37, further comprising one or more hook assemblies, wherein each of the one or more hook assemblies is attached to the first sidearm or the second sidearm, and wherein each of the one or more hook assemblies comprises: a hook comprising a hook shank and a hook bend; a linear rail; and a sliding block configured to slide along the linear rail; wherein the hook shank is attached to the sliding block.
Example 39 is a system as in any of Examples 21-38, wherein each of the one or more hook assemblies further comprises a hook cutout configured to receive the hook shank, wherein the hook cutout is cut into a sidewall of one or more of the first sidearm or the second sidearm.
Example 40 is a system as in any of Examples 21-39, wherein each of the one or more hook assemblies further comprises: a first magnet attached to the linear rail at a proximal end of the linear rail, wherein the proximal end of the linear rail is disposed nearer to the thermal cooling system; and one or more of a second magnet or a magnetic material attached to the hook shank at a proximal end of the hook shank, wherein the proximal end of the hook shank is disposed nearer to the thermal cooling system; wherein the first magnet does not engage with the one or more of the second magnet or the magnetic material when the sliding block is located at a distal end of the linear rail; and wherein the first magnet engages with the one or more of the second magnet or the magnetic material when the sliding block is located at the proximal end of the linear rail.

In the foregoing Detailed Description of the Disclosure, various features of the disclosure are grouped together in a single embodiment for the purpose of streamlining the disclosure. This method of disclosure is not to be interpreted as reflecting an intention that the claimed disclosure requires more features than are expressly recited in each claim. Rather, inventive aspects lie in less than all features of a single foregoing disclosed embodiment.

It is to be understood that any features of the above-described arrangements, examples, and embodiments may be combined in a single embodiment comprising a combination of features taken from any of the disclosed arrangements, examples, and embodiments. It is to be understood that the above-described arrangements are only illustrative of the application of the principles of the disclosure. Numerous modifications and alternative arrangements may be devised by those skilled in the art without departing from the spirit and scope of the disclosure and the appended claims are intended to cover such modifications and arrangements.

Thus, while the disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

The foregoing description has been presented for the purposes of illustration and description. It is not intended to be exhaustive or to limit the disclosure to the precise form disclosed. Many modifications and variations are possible in light of the above teaching. Further, it should be noted that any or all the aforementioned alternate implementations may be used in any combination desired to form additional hybrid implementations of the disclosure.

1. A system for thermal cooling within a surgeon control console of a robotic surgical system, the system comprising:
a housing of the surgeon control console, wherein the housing defines an interior space; and
a thermal cooling system disposed within the interior space defined by the housing, wherein the thermal cooling system comprises:
a first haptic interface device component;
a processing unit;
a second haptic interface device component;
a heat sink that receives heat generated by the processing unit during operation; and
a plurality of fans;
wherein the plurality of fans is configured to pull air into the interior space, through the first haptic interface device component, through the heat sink, and through the second haptic interface device component.

2. The system of claim 1, wherein the interior space defined by the housing is disposed within an armrest console of the surgeon control console, and wherein the surgeon control console provides data to the robotic surgical system that results in the robotic surgical system moving one or more robotic arms or surgical instruments.

3. The system of claim 1, wherein the processing unit comprises a central processing unit (CPU) and a graphics processing unit (GPU).

4. The system of claim 3, wherein the heat sink comprises:
a first heat sink located adjacent to the CPU, wherein the first heat sink primarily receives heat generated by the CPU during operation; and
a second heat sink located adjacent to the GPU, wherein the second heat sink primarily receives heat generated by the GPU during operation.

5. The system of claim 4, wherein the first heat sink comprises a first plurality of heat sink specifications comprising:
a first plurality of fins, wherein the first heat sink comprises a first quantity of the first plurality of fins;
a first fin width, wherein at least a portion of the first plurality of fins has the first fin width; and
a first fin spacing, wherein two or more of the first plurality of fins is spaced apart according to the first fin spacing; and
wherein the second heat sink comprises a second plurality of heat sink specifications comprising:
a second plurality of fins, wherein the second heat sink comprises a second quantity of the second plurality of fins;
a second fin width, wherein at least a portion of the second plurality of fins has the second fin width; and
a second fin spacing, wherein two or more of the second plurality of fins is spaced apart according to the second fin spacing; and
wherein at least one of the first plurality of heat sink specifications is different from a corresponding specification of the second plurality of heat sink specifications.

6. The system of claim 5, wherein the first fin spacing is different than the second fin spacing.

7. The system of claim 1, and wherein the interior space defined by the housing comprises:
a first side and a second side, wherein the second side is located opposite to the first side;
a first vent disposed at the first side, wherein a first fan of the plurality of fans is configured to pull the air into the interior space by way of the first vent;
the first haptic interface device component disposed at the first side;
the second haptic interface device component disposed at the second side; and
a second vent disposed at the second side, wherein a discharge fan of the plurality of fans is configured to pull heated air out of the interior space by way of the second vent.

8. The system of claim 7, wherein the processing unit and the heat sink are disposed in between the first haptic interface device component and the second haptic interface component such that an airflow pathway for the thermal cooling system comprises:
the air entering the interior space through the first vent;
the air passing through the first haptic interface device component to generate heated air;
the heated air passing through the heat sink after passing through the first haptic interface device component;
the heated air passing through the second haptic interface device component after passing through the heat sink; and
the heated air exiting the interior space through the second vent after passing through the second haptic interface device component.

9. The system of claim 8, wherein the processing unit and the heat sink comprises:
a central processing unit (CPU) and a first heat sink disposed above the CPU; and
a graphics processing unit (GPU) and a second heat sink disposed above the GPU;
wherein the heated air passing through the heat sink comprises the heated air passing through the first heat sink disposed above the CPU or the second heat sink disposed above the GPU.

10. The system of claim 8, further comprising a baffle disposed within the interior space defined by the housing, wherein the baffle prevents the air pulled into the interior space from moving to the second side prior to passing through the heat sink.

11. The system of claim 1, wherein the plurality of fans comprises:
a first plurality of haptic interface device component fans associated with the first haptic interface device component; and
a second plurality of haptic interface device component fans associated with the second haptic interface device component.

12. The system of claim 11, wherein the plurality of fans further comprises:
a central processing unit (CPU) fan configured to pull air into a first heat sink disposed above a CPU;
a graphics processing unit (GPU) fan configured to pull air into a second heat sink disposed above a GPU; and
a discharge fan configured to pull heated air out of the interior space by way of a vent cut into a wall of the housing.

13. The system of claim 1, wherein one of more of the first haptic interface device component or the second haptic interface device component comprises a processor in communication with a surgical haptic interface device;
wherein the surgical haptic interface device is disposed outside the interior space defined by the housing; and
wherein the processor in communication with the surgical haptic interface device is configured to translate movements of the surgical haptic interface device into robotic movements executed by the robotic surgical system.

14. The system of claim 1, wherein the robotic surgical system comprises:
a robotic system comprising a robotic arm and a surgical instrument attached to the robotic arm;
the surgeon control console in communication with the robotic system, wherein the surgeon control console provides data to the robotic system, and wherein the robotic system utilizes the data to calculate one or more movements to be executed by the robotic arm or the surgical instrument; and
a visualization system comprising a camera and a light source.

15. The system of claim 1, wherein the housing is a component of an upper platform of an armrest console, and wherein the armrest console further comprises:
an armrest disposed substantially parallel to the upper platform;
a first sidearm attached to the upper platform and the armrest, wherein the first sidearm is substantially perpendicular to the upper platform and the armrest; and
a second sidearm attached to the upper platform and the armrest, wherein the second sidearm is disposed substantially parallel to the first sidearm.

16. The system of claim 15, further comprising one or more hook assemblies, wherein each of the one or more hook assemblies is attached to the first sidearm or the second sidearm, and wherein each of the one or more hook assemblies comprises:
a hook comprising a hook shank and a hook bend;
a linear rail; and
a sliding block configured to slide along the linear rail;
wherein the hook shank is attached to the sliding block.

17. The system of claim 16, wherein each of the one or more hook assemblies further comprises a hook cutout configured to receive the hook shank, wherein the hook cutout is cut into a sidewall of one or more of the first sidearm or the second sidearm.

18. The system of claim 17, wherein each of the one or more hook assemblies further comprises:
a first magnet attached to the linear rail at a proximal end of the linear rail, wherein the proximal end of the linear rail is disposed nearer to the thermal cooling system; and
one or more of a second magnet or a magnetic material attached to the hook shank at a proximal end of the hook shank, wherein the proximal end of the hook shank is disposed nearer to the thermal cooling system;
wherein the first magnet does not engage with the one or more of the second magnet or the magnetic material when the sliding block is located at a distal end of the linear rail; and
wherein the first magnet engages with the one or more of the second magnet or the magnetic material when the sliding block is located at the proximal end of the linear rail.

19. A surgeon control console comprising:
an armrest console defining an interior space; and
a thermal cooling system disposed within the interior space defined by the armrest console, the thermal cooling system comprising:
a first haptic interface device component;
a processing unit;
a second haptic interface device component;
a heat sink that receives heat generated by the processing unit during operation; and
a plurality of fans configured to pull air into the interior space, through the first haptic interface device component, through the heat sink, and through the second haptic interface device component.

20. A robotic surgical system comprising:
a robotic system comprising a robotic arm configured to attach to a surgical instrument;
a surgeon control console configured to provide data to the robotic system, the surgeon control console comprising:
a housing defining an interior space; and
a first haptic interface device component disposed within the interior space;
a processing unit disposed within the interior space;
a second haptic interface device component disposed within the interior space;
a heat sink that receives heat generated by the processing unit during operation, the heat sink being disposed within the interior space; and
a plurality of fans configured to pull air into the interior space, through the first haptic interface device component, through the heat sink, and through the second haptic interface device component.
